(19) United States
(12) Patent Application Publication
(10) Pub. No.: US 2008/0280853 A1
(43) Pub. Date: Nov. 13, 2008

(54) COMPOSITIONS FOR REDUCING, AMELIORATING, TREATING, OR PREVENTING CONDITION OF DRY EYE AND METHODS OF MAKING AND USING SAME

(76) Inventors: Erning Xia, Penfield, NY (US); Dharmendra M. Jani, Fairport, NY (US)

Correspondence Address: Bausch & Lomb Incorporated, One Bausch & Lomb Place, Rochester, NY, 14604-2701, US

(21) Appl. No.: 12/114,878

(22) Filed: May 5, 2008

Related U.S. Application Data
(60) Provisional application No. 60/916,326, filed on May 7, 2007.

(51) Int. Cl. A61K 31/715; A61P 27/02

(52) U.S. Cl. 514/54

(57) ABSTRACT

Compositions for relief, treatment, or prevention of a condition of dry eye comprise alginate, at least a polyol, and a pharmaceutically acceptable carrier, wherein the compositions have pH in the range from about 5 to about 7.5. In some embodiment, the compositions can further include a chelating agent, a buffering system or agent, an organic acid, or combinations thereof.

### CROSS REFERENCE

This application claims the benefit of Provisional Patent Application No. 60/916,326 filed May 7, 2007 which is incorporated by reference herein.

### BACKGROUND

The present invention relates to compositions for reducing, ameliorating, treating, or preventing condition of dry eye, and methods of making and using such compositions. In particular, the present invention relates to compositions and methods for reducing, ameliorating, treating, or preventing discomfort of dry eye condition.

Dry eye, also known as keratoconjunctivitis sicca or dyslacrima, is a common opthalmological disorder affecting millions of people. A patient with dry eye may experience burning, a feeling of dryness, and persistent irritation. In severe cases, dry eye can seriously impair a person's vision and hence handicap the sufferer in activities such as driving. Certain diseases such as Sjogren's disease manifest dry eye symptoms. Also, as people age, the lacrimal glands in the eye may produce less moisture, resulting in eyes that become dry, inflamed, itchy, and gritty.

Although it appears that dry eye may result from a variety of unrelated pathogenic causes, all presentations of the condition share a common feature, namely the breakdown of the precorneal tear film, which breakdown commonly results in dehydration of the exposed outer ocular surface and hence the symptoms described above.

A number of approaches exist for the treatment of dry eye. One common approach has been to supplement the ocular tear film using artificial tears instilled throughout the day. Examples of the tear substitute approach include the use of buffered, isotonic saline solutions and aqueous solutions containing water-soluble polymers that render the solutions more viscous and thus less easily shed by the eye by the washing action of the tear fluid. See, for example, U.S. Pat. No. 5,209,927 to Gressel et al.; U.S. Pat. No. 5,294,607 to Glonek et al.; and U.S. Pat. No. 4,409,205 to Shively;

Although these approaches have met with some success in some cases, significant challenges in the treatment of dry eye nevertheless remain. Problems include the fact that the use of tear substitutes, while temporarily effective, generally requires repeated application over the course of a patient's waking hours, not uncommonly ten or more times over the course of a day. Such an approach is inconvenient to a patient. Although increasing the viscosity of the dry-eye product may extend the product's duration in the eye, increase in viscosity is effective at extending duration only to a limited extent. Viscous ophthalmic drops are sometimes undesirable because they feel sticky in the eye. Further, increases in the duration of the product would be highly desirable.

Alginate, for the purpose of this application is a polysaccharide that comprises monomeric units of β-D-mannuronic acid and α-L-guluronic acid, or salts thereof, or derivatives of such acids or salts.

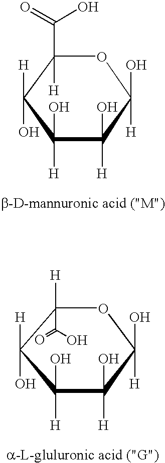

Some alginate polymers are block copolymers with blocks of the guluronic acid (or a salt thereof) monomeric units alternating with blocks of the mannuronic acid (or a salt thereof) monomeric units. Other alginate molecules have alternating single monomeric units of guluronic acid (or a salt thereof) and mannuronic acid (or a salt thereof). The ratio and distribution of the M and G components along with the average molecular weight affect the physical and chemical properties of the copolymer. See A. Haug et al., Acta Chem. Scand., Vol. 20, 183-190 (1966). Alginate polymers have viscoelastic Theological properties and other properties that make it suitable for some medical applications. See G. Klock et al., “Biocompatibility of Mannuronic Acid-Rich Alginates,” Biomaterials, Vol. 18, No. 10, 707-713 (1997).

The use of alginate as a thickener for topical ophthalmic use is disclosed in U.S. Pat. No. 6,528,465 and U.S. Patent Application Publication 2003/0232089. U.S. Pat. No. 5,776,445 discloses the use of alginate as a drug delivery agent that is topically applied to the eye. Particularly, the amount of guluronic acid in the alginate was taught to exceed 50%.

U.S. Patent Application Publication 2003/0232089 teaches a dry-eye formulation that contains two polymer ingredients including alginate.

Ophthalmic compositions typically include other ingredients that provide additional properties. For example, polyols (e.g., glycerin) are known as demulcents and tonicity adjusting agents in ophthalmic formulations including formulations for the delivery of an active pharmaceutical agent. See; e.g., U.S. Pat. Nos. 5,075,104 and 5,209,927, which teach the use of a polyol with a cabomer polymer.

In addition, pharmaceutical compositions, including those for ophthalmic applications, very often include an antimicrobial preservative to allow for multiple uses. Some common preservatives that have been used in ophthalmic formulations include benzalkonium chloride, chlorobutanol, alexidine, chlorhexidine, hexamethylene biguanides, quaternary ammonium compounds, and parabens. See; e.g., U.S. Pat. Nos. 6,833,358; 6,852,311; 6,960,575; and 7,105,473. However, these preservatives can result in some discomfort to sensitive patients, especially those who already suffer from dry eye condition.

Therefore, in view of the shortcomings of prior-art compositions, there is a continued need to provide improved compositions for the reduction, amelioration, treatment, or prevention of the discomfort resulting from the dry eye condition. It is also desirable to provide such compositions that are gentle to the ocular surface.

### SUMMARY

In general, the present invention provides a composition that is capable of reducing, ameliorating, treating, or preventing discomfort resulting from a condition of dry eye.

In one aspect, the composition has lower risk of introducing unwanted exogenous side effects, such as an unwanted sensation. Alternatively, the composition is gentle to the ocular surface.

In another aspect, a composition of the present invention comprises: (a) alginate; and (b) at least a polyol; wherein the composition has a pH in a range from about 5 to about 7.5.

In still another aspect, the polyol has 2 to 18 (or, alternatively, 2 to 12, or 2 to 10, or 2 to 6, or 2 to 4) carbon atoms.

In yet another aspect, the composition is free or essentially free of preservatives.

In a further aspect, the present invention also provides a method of reducing, ameliorating, treating, or preventing a condition of dry eye. The method comprises administering to an eye of a subject suffering from such a condition any one of the compositions herein disclosed.

In still another aspect, such a composition comprises a solution, a dispersion, an emulsion (such as oil-in-water emulsion), a gelable composition, or a gel.

In yet another aspect, the present invention provides a method for preparing a pharmaceutical composition. The method comprises combining alginate, a polyol, and a pharmaceutically acceptable carrier to form a mixture having a pH in a range from about 5 to about 7.5.

Other features and advantages of the present invention will become apparent from the following detailed description and claims.

### DETAILED DESCRIPTION

In general, the present invention provides a composition that is capable of reducing, ameliorating, treating, or preventing discomfort resulting from a dry eye condition.

In one aspect, the composition has lower risk of introducing unwanted exogenous side effects, such as an unwanted irritating, burning, or stinging sensation. Alternatively, the composition is gentle to the ocular surface.

In another aspect, a composition of the present invention comprises: (a) alginate; and (b) a polyol; wherein the composition has a pH in a range from about 5 to about 7.5.

In still another aspect, a composition of the present invention comprises: (a) alginate; (b) at least a polyol; and (c) a pharmaceutically acceptable carrier; wherein the composition has a pH in a range from about 5 to about 7.5. In one embodiment, the pH of a composition of the present invention is in the range from about 5.5 to about 7.5. In another embodiment, the composition has a pH in the range from about 6 to about 7.5 (or alternatively, from about 6 to about 7, or from about 5.5 to about 7, or from about 5.5 to about 6.5, or from about 5 to about 6.8, or from about 5.5 to about 6.8).

In yet another aspect, said alginate is present in an amount from about 0.01 to about 2 percent by weight of the total composition. Alternatively, said alginate is present in an amount from about 0.01 to about 1 percent by weight (or from about 0.01 to about 0.5, or from about 0.1 to about 1, or from about 0.1 to about 0.5, or from about 0.1 to about 0.3 percent by weight) of the total composition.

In one embodiment, said alginate comprises alternating homopolymeric blocks, each comprising or consisting of monomeric units of mannuronic acid (or a salt thereof) (“M”) or guluronic acid (or a salt thereof) (“G”). In another embodiment, said alginate comprises alternating single units of M and G.

In certain embodiments, said alginate has a molecular weight in a range from about 50 kDa to about 5000 kDa. Alternatively, said alginate has a molecular weight in a range from about 50 kDa to about 2000 kDa (or from about 50 kDa to about 1000 kDa, or from about 50 kDa to about 700 kDa, from about 50 kDa to about 500 kDa, or from about 50 kDa to about 100 kDa, or from about 100 kDa to about 2000 kDa, or from about 100 kDa to about 1000 kDa, or from about 100 kDa to about 500 kDa, or from about 500 kDa to about 2000 kDa, or from about 500 kDa to about 1000 kDa). Suitable alginates are known under the trade name Protanal, available from FMC BioPolymer, Philadelphia, Pa.

In one preferred embodiment, the molecular weight is about 200-300 kDa.

The proportion of G monomeric units in an alginate molecule suitable for a composition of the present invention can be in the range from about 10 to about 90 percent of the total number of monomeric units of the alginate molecule. Alternatively, such proportion can be in the range from about 20 to about 75 (or from 30 to about 60, or from about 25 to about 50, or from about 20 to about 50, or from about 10 to about 30) percent of the total number of monomeric units of the alginate molecule. In one embodiment, such proportion is about 35-45 percent.

Polyols suitable for use in a composition of the present invention include those having 2 to 18 (or, alternatively, 2 to 12, or 2 to 10, or 2 to 6, or 2 to 4) carbon atoms. In one embodiment, the polyol contains 2 to 6 carbon atoms. In another embodiment, the polyol contains 2 to 6 carbon atoms. Non-limiting examples of suitable polyols include glycerin, ethylene glycol, propylene glycol, sorbitol, mannitol, xylitol, monosaccharides, disaccharides, trisaccharides, and combinations thereof. In one embodiment, the polyol is selected from the group consisting of glycerin, ethylene glycol, propylene glycol, sorbitol, mannitol, xylitol, monosaccharides, and combinations thereof. In another embodiment, the polyol is selected from the group consisting of disaccharides. In one preferred embodiment, the polyol is a combination of glycerin and propylene glycol.

The concentration of a polyol included in a composition of the present invention is in a range from about 0.01 to about 5 percent by weight of the total composition. Alternatively, the concentration of a polyol is in a range from about 0.01 to about 2 percent (or from about 0.01 to about 1, or from about 0.01 to about 0.5, or from about 0.05 to about 1, or from about 0.05 to about 0.5, or from about 0.1 to about 1, or from about 0.1 to about 0.5, or from about 0.1 to about 0.3, or from about 0.2 to about 1 percent) by weight of the total composition.

In another aspect, the ratio of alginate to polyol is in a range from about 1:20 to about 20:1. Alternatively, the ration is in a range from about 1:10 to about 10:1, or from about 1:7 to about 7:1, or from about 1:5 to about 5:1, or from about 1:3 to about 3:1.

In yet another aspect, a composition of the present invention further comprises an organic acid. Non-limiting examples of such an organic acid includes sorbic acid, acetic acid, dehydroacetic acid, proprionic acid, butyric acid, isobutyric acid, valeric acid, hexanoic acid (caproic acid), heptanoic acid (enanthic acid), octanoic acid (caprylic acid), nonanoic acid (pelargonic acid), decanoic acid (capric acid), (+) camphoric acid, peroxyacetic acid, n-peroxybutyric acid, peroxyformic acid, peroxypropionic acid, malonic acid, dimethylmalonic acid, succinic acid, glutaric acid, β-methylglutaric acid, adipic acid, pimelic acid, suberic acid, azelaic acid, 1,1-cyclopentanediacetic acid, 1,2-trans-cyclopentanedicarboxylic acid, 1,3-trans-cyclopentanedicarboxylic acid, 1,3-trans-cyclohexanedicarboxylic acid, 1,4-cis-cyclohexanedicarboxylic acid, cyclohexanecarboxylic acid, benzoic acid, methoxybenzoic acid, p-n-propoxybenzoic acid, p-n-butoxybenzoic acid, and combinations thereof. Their pKa values are shown in Table 1. If desired, the organic acid can be chosen to provide preservative efficacy.

TABLE 1
pKa Values of Some Organic Acids

| Acid Name | pKa |
|---|---|
| sorbic acid | 4.8 |
| acetic acid | 4.76 |
| dehydroacetic acid | 5.40 |
| propionic acid | 4.87 |
| butyric acid | 4.85 |
| isobutyric acid | 4.84 |
| valeric acid | 4.85 |
| hexanoic acid | 4.8 |
| heptanoic acid | 4.89 |
| octanoic acid | 4.89 |
| nonanoic acid | 4.95 |
| decanoic acid | 4.9 |
| (+) camphoric acid | 4.72 |
| peroxyacetic acid | 8.2 |
| n-peroxybutyric acid | 8.2 |
| peroxyformic acid | 7.1 |
| peroxypropionic acid | 8.1 |
| malonic acid | 2.83, 5.69 |
| dimethylmalonic acid | 3.17, 6.06 |
| succinic acid | 4.19, 5.48 |
| glutaric acid | 4.34, 5.42 |
| β-methylglutaric acid | 4.25, 6.22 |
| adipic acid | 4.42, 5.41 |
| pimelic acid | 4.48, 5.42 |
| suberic acid | 4.52, 5.4 |
| azelaic acid | 4.55, 5.41 |
| 1,1-cyclopentanediacetic acid | 3.82, 6.70 |
| 1,2-trans- | 3.89, 5.91 |
| cyclopentanedicarboxylic acid | |
| 1,3-trans- | 4.40, 5.45 |
| cyclopentanedicarboxylic acid | |
| 1,3-trans-cyclohexanedicarboxylic | 4.18, 5.93 |
| acid | |
| 1,4-cis-cyclohexanedicarboxylic | 4.44, 5.79 |
| acid | |
| cyclohexanecarboxylic acid | 4.90 |
| benzoic acid | 4.21 |
| p-methoxybenzoic acid | 4.47 |
| p-n-propoxybenzoic acid | 4.46 |
| p-n-butoxybenzoic acid | 4.53 |

In some embodiments, said organic acid is selected from the group consisting of sorbic acid, acetic acid, propionic acid, peroxyacetic acid, peroxypropionic acid, peroxyformic acid, cyclohexanecarboxylic acid, and combinations thereof.

In some other embodiments, said organic acid is selected from the group consisting of sorbic acid, acetic acid, dehydroacetic acid, propionic acid, peroxyacetic acid, peroxypropionic acid, and combinations thereof.

In still some other embodiments, said organic acid is selected from the group consisting of succinic acid, glutaric acid, β-methylglutaric acid, adipic acid, pimelic acid, suberic acid, azelaic acid, 1,1-cyclopentanediacetic acid, 1,2-trans-cyclopentanedicarboxylic acid, 1,3-trans-cyclopentanedicarboxylic acid, 1,3-trans-cyclohexanedicarboxylic acid, 1,4-cis-cyclohexanedicarboxylic acid, and combinations thereof.

In still another aspect, said organic acid is present in a composition of the present invention at a concentration in a range from about 0.01 to about 2 percent by weight of the total composition. Alternatively, said organic acid is present in a composition of the present invention at a concentration in a range from about 0.01 to about 1 percent (or from about 0.01 to about 0.5, or from about 0.05 to about 0.5, or from about 0.05 to about 0.3, or from about 0.1 to about 0.5, or from about 0.1 to about 0.3 percent) by weight of the total composition.

In still another aspect, said organic acid has a pKa that is no more than about 1.5 units less than the pH of the composition. Alternatively, said pKa is no more than about 1 unit less than the pH of the composition. In still another embodiment, said pKa is no more than about 0.5 unit less than the pH of the composition. In one embodiment, said organic acid is a monocarboxylic acid.

In another embodiment, the alginate-containing composition is characterized in that it has a Mark-Houwink number that is a minimum of about 0.6. Typically, the Mark-Houwink number is desirably in a range from about 0.6 to about 1.2. In one embodiment, the Mark-Houwink number is about 1.

A composition is analyzed using size exclusion chromatography (SEC) with triple detection. Particularly, lights scattering, viscometry trace, and refractive index detection analysis are performed. The Mark-Houwink number is calculated from the data obtained from the triple detection SEC method using the mathematical technique disclosed in “Introduction to Physical Polymer Science,” Third Edition, L. H. Sperling, Wiley-Interscience, John Wiley & Sons, Inc., New York, 2001. The shape of alginate particles in the composition may be inferred from the Mark-Houwink number as indicated in Table 2.

TABLE 2
Values of the Mark-Houwink number

| Mark-Houwink number | Interpretation |
|---|---|
| 0 | spheres |
| 0.5-0.8 | random coils |
| 1.0 | stiff coils |
| 2.0 | rods |

In yet another aspect, a composition of the present invention is free of alexidine, chlorhexidine, parabens, benzalkonium chloride, polymeric quaternary ammonium compounds, and derivatives thereof.

The aqueous solutions employed in this invention may contain one or more additional ingredients that are commonly present in ophthalmic solutions, for example, tonicity-adjusting agents, buffers, antioxidants, viscosity-adjusting agents, surfactants, stabilizers, chelating agents, and the like, which aid in making ophthalmic compositions more comfortable to the user.

A composition of the present invention can be adjusted with tonicity-adjusting agents to approximate the tonicity of normal lacrimal fluids that is equivalent to a 0.9 percent (by weight) solution of sodium chloride or a 2.8 percent (by weight) of glycerin solution. The compositions of the present invention desirably have osmolality in a range from about 200 mOsm/kg to about 400 mOsm/ka. Alternatively, the osmolality is in the range from about 220 to about 360 mOsm/kg (or from about 220 to about 320 mOsm/kg, or from about 240 to about 300 mOsm/kg, or from about 240 to about 280 mOsm/kg, or from about 220 to about 280 mOsm/kg, or from about 220 to about 260 mOsm/kg, or from about 200 to about 300 mOsm/kg).

In another aspect, a composition of the present invention can comprise a buffering agent or system. Suitable buffers for use in compositions of the present invention include Good's buffers. Non-limiting examples of buffering agents include MES (2-(N-morpholino)ethanesulfonic acid hemisodium salt) having pKa of 6.1 at 25° C. and pH in the range of about 5.5-6.7; HEPES (N-{2-hydroxyethyl}peperazine-N′-{2-ethanesulfonic acid}) having pKₐ of 7.5 at 25° C. and pH in the range of about 6.8-8.2; BES (N,N-bis{2-hydroxyethyl}2-aminoethanesulfonic acid) having pKₐ of 7.1 at 25° C. and pH in the range of about 6.4-7.8; MOPS (3-{N-morpholino}propanesulfonic acid) having pKₐ of 7.2 at 25° C. and pH in the range of about 6.5-7.9; BIS-TRIS (bis(2-hydroxyethyl)amino-tris(hydroxymethyl)methane) having pKa of 6.5 at 25° C. and pH in the range of about 5.8-7.2; citrate buffer (pH in the range of about 5.5-7.2); maleate buffer (pH in the range of about 5.5-7.2); succinate buffer (pH in the range of about 5.5-6.5); malate buffer (pH in the range of about 4-6); and boric acid/sodium borate buffer (pH in the range of about 7-9). Other pharmaceutically acceptable buffers that provide pH in the range of 5 to 7.5 also can be used. In one embodiment, the buffer system comprises boric acid and sodium borate.

A composition of the present invention can have a viscosity in the range from about 5 to about 100,000 centipoise (“cP”) or mPa·s (or alternatively, from about 10 to about 50,000, or from about 10 to about 20,000, or from about 10 to about 10,000, or from about 10 to about 1,000, or from about 100 to about 10,000, or from about 100 to about 20,000, or from about 100 to about 50,000 or from about 500 to about 10,000, or from about 500 to about 20,000 cP or mPa·s).

The use of viscosity enhancing agents to provide the compositions of the invention with viscosities greater than the viscosity of simple aqueous solutions may be desirable to increase the retention time in the eye. Such viscosity enhancing agents include, for example, polyvinyl alcohol, polyvinyl pyrrolidone, methyl cellulose, hydroxypropylmethyl cellulose, hydroxyethyl cellulose, carboxymethyl cellulose, hydroxypropyl cellulose or other agents known to those skilled in the art. Such agents are typically employed at a level of from 0.01 to 10 percent (alternatively, 0.1 to 5 percent, or 0.1 to 2 percent) by weight.

Suitable surfactants include polyvinyl pyrrolidone, polyvinyl alcohol, polyethylene glycol, ethylene glycol, and propylene glycol. Other surfactants are polysorbates (such as polysorbate 80 (polyoxyethylene sorbitan monooleate), polysorbate 60 (polyoxyethylene sorbitan monostearate), polysorbate 20 (polyoxyethylene sorbitan monolaurate), commonly known by their trade names of Tween® 80, Tween® 60, Tween® 20), poloxamers (synthetic block polymers of ethylene oxide and propylene oxide, such as those commonly known by their trade names of Pluronic®; e.g., Pluronic® F127 or Pluronic® F108)), or poloxamines (synthetic block polymers of ethylene oxide and propylene oxide attached to ethylene diamine, such as those commonly known by their trade names of Tetronic®; e.g., Tetronic® 1508 or Tetronic® 908, etc., other nonionic surfactants such as Brij®, Myrj®, and long chain fatty alcohols (i.e., oleyl alcohol, stearyl alcohol, myristyl alcohol, docosohexanoyl alcohol, etc.) with carbon chains having about 12 or more carbon atoms (e.g., such as from about 12 to about 24 carbon atoms). A surfactant helps a topical formulation to spread on the ocular surface.

Suitable antioxidants include, but are not limited to, ascorbic acid and its esters, sodium bisulfite, butylated hydroxytoluene, butylated hydroxyanisole, tocopherols, and combinations thereof. Antioxidants can be included in a composition of the present invention in an amount in the range from about 0.005 to about 0.05 percent by weight (or alternatively, from about 0.005 to about 0.02 percent, or from about 0.005 to about 0.01 percent, by weight).

Suitable chelating agents include, but are not limited to, hydroxyalkylphosphonic acids and polyaminocarboxylic acids (such as ethylenediaminetetraacetic acid (“EDTA”), diethylenetriaminepentaacetic acid (“DTPA”), nitrilotriacetic acid (“NTA”), hexamethylenediaminetetraacetic acid (“HMDTA”), N-(2-hydroxyethyl)ethylenediamine-N,N′,N′-triacetic acid (“HEEDTA” or HEDTA”), hydroxymethylethylenediaminetriacetic acid (“HMEDTA”), 1,3-diamino-2-propanol-N,N,N′,N′-tetracetic acid, 1,3-diamino-2-propane-N,N,N′,N′-tetracetic acid, ethylene glycol-bis(2-aminoethylether)-N,N,N′,N′-tetraacetic acid, ethylenediamine-N,N-diacetic acid (“EDDA”), nicotinic acid, deoxymugineic acid (“DMA”), 3,6,9-triaza-12-oxa-3,6,9-tricarboxymethylene-10-carboxy-13-phenyl-tridecanoic acid (“B-19036”), and combinations thereof). Other non-limiting examples of chelating agents include cyclic aminocarboxylic acids, such as 1,4,7,10-tetraazacyclododecane-N,N′,N″,N′″-tetraacetic acid (“DOTA”), p-isothiocyanatobenzyl-1,4,7,10-tetraazacyclododecane-1,4,7,10-tetraacetic acid (“p-SCN-Bz-DOTA”), 1,4,7,10-tetraazacyclododecane-N,N′,N″-triacetic acid (“DO3A”), 1,4,7,10-tetraazacyclododecane-1,4,7,10-tetrakis(2-propionic acid) (“DOTMA”), 1,4,7-triazacyclononane-N,N′,N″-triacetic acid (“NOTA”), 1,4,8,11-tetraazacyclotetradecane-N,N′,N″,N′″-tetraacetic acid (“TETA”), triethylenetetraaminehexaacetic acid (“TTHA”), trans-1,2-diaminohexanetetraacetic acid (“CYDTA”), 1,4,7,10-tetraazacyclododecane-1-(2-hydroxypropyl)-4,7,10-triacetic acid (“HP-DO3A”), trans-cyclohexanediaminetetraacetic acid (“CDTA”), trans(1,2)-cyclohexanediethylenetriaminepentaacetic acid (“CDTPA”), 1-oxa-4,7,10-triazacyclododecane-N,N′,N″-triacetic acid (“OTTA”), 1,4,7,10-tetraazacyclododecane-1,4,7,10-tetrakis {3-(4-carboxyl)-butanoic acid}, 1,4,7,10-tetraazacyclododecane-1,4,7,10-tetrakis(acetic acid-methyl amide), 1,4,7,10-tetraazacyclododecane-1,4,7,10-tetrakis(methylene phosphonic acid). Chelating agents can be included in a composition of the present invention in an amount in the range from about 0.005 to about 0.2 percent by weight (or alternatively, from about 0.005 to about 0.1, from about 0.005 to about 0.05 percent, or from about 0.005 to about 0.02 percent, by weight).

The present invention also provides a method of ameliorating, reducing, treating, or preventing a condition of dry eye. The method comprises administering to an affected eye a composition that comprises: (a) alginate; (b) a polyol; and (c) a pharmaceutically acceptable carrier; wherein the composition has a pH in a range from about 5 to about 7.5. In one embodiment, the composition has a pH in the range from about 5.5 to about 7.5. In another embodiment, the composition has a pH in the range from about 6 to about 7.5 (or alternatively, from about 6 to about 7, or from about 5.5 to about 7, or from about 5.5 to about 6.5, or from about 6.5 to about 7.5).

In one embodiment, the composition further comprises an organic acid. In another embodiment, the organic provides preservative efficacy to the composition.

In one aspect, the various ingredients of the composition are present in amounts disclosed herein.

In another aspect, the composition can be applied in one or more drops to an ocular surface once per day, twice per day, or three or more times per day, as needed.

In still another aspect, the method provides relief to an ocular discomfort resulting from a dry eye condition.

In a further aspect, the present invention provides a method for producing a composition for ameliorating, reducing, treating, or preventing a condition of dry eye. The method comprises combining: (1) alginate; (2) at least a polyol; and (3) a pharmaceutically acceptable carrier, to form a mixture; wherein a pH of the mixture has a value in a range from about 5 to about 7.5 (or alternatively, from about 5 to about 7, or from about 5.5 to about 7, or from about 5 to about 6, or from about 5.5. to about 6.5); and said mixture comprises said composition.

In still another aspect, the step of combining further includes adding a chelating agent into said mixture. Suitable chelating agents and their concentrations are disclosed herein above.

In yet another aspect, the method further comprises: (b) adjusting the pH value of the mixture to bring it into said pH range.

In a further aspect, the method further comprises: (c) subjecting the mixture to a sterilization procedure. In one embodiment, the sterilization procedure can comprise exposing the mixture to α, β, or γ radiation; autoclaving the mixture; or heating the mixture to a temperature in arrange from about 100 to about 125° C., for 10 minutes or longer, but less than a time that would result in a degradation of the alginate.

A composition of the present invention may be packaged in unit-dose (for single use) or multi-dose (for multiple use) containers.

Table 3 shows exemplary compositions of the present invention. The composition of Example 1 has been prepared and found to be capable of providing relief to the dry eye condition.

TABLE 3
Some Compositions for Dry Eye Condition

| | Example | | | | |
|---|---|---|---|---|---|
| Ingredient | 1 | 2 | 3 | 4 | 5 |
| Boric acid NF | 0.5 | 0.3 | 0.6 | 0.2 | 0.5 |
| (wt. %) | | | | | |
| Sodium borate | 0.014 | 0.03 | 0.01 | 0.04 | 0.04 |
| NF (wt. %) | | | | | |
| Alginate⁽¹⁾ | 0.25 | 0.25 | 0.4 | 0.5 | 0.5 |
| (wt. %) | | | | | |
| Glycerin | 0.6 | 0.6 | 1 | 1 | 0.6 |
| (wt. %) | | | | | |
| Propylene | 0.6 | 0.6 | 0 | 0 | 0.6 |
| glycol | | | | | |
| (wt. %) | | | | | |
| HAP⁽²⁾ (wt. %) | 0.05 | 0.02 | 0.1 | 0.15 | 0.2 |
| Purified water | q.s. 100 | q.s. 100 | q.s. 100 | q.s. 100 | q.s. 100 |
| pH | 6.7-7.1 | — | — | — | — |
| Osmolality | 200-240 | ~200-300 | ~200-300 | ~200-300 | ~200-300 |
| (mOsm/kg) | | (expected) | (expected) | (expected) | (expected) |

Notes:
⁽¹⁾Protanal LF 200M, sodium alginate
⁽²⁾Hydroxyalkylphosphonic acid

Table 4 shows some other exemplary compositions within the scope of the present invention that have not been experimentally prepared. These compositions are expected to have utility in providing relief to a dry eye condition.

TABLE 4
Some Other Compositions for Dry Eye Condition

| | Example | | | |
|---|---|---|---|---|
| Type of | 6 | | 7 | |
| Ingredient | Ingredient | (wt. %) | Ingredient | (wt. %) |
| Buffer | MES | 1 | Succinate | 1 |
| Alginate | Protanal LF | 0.3 | Protanal LF | 0.4 |
| | 240D⁽³⁾ | | 240D⁽³⁾ | |
| Polyol | Glycerin | 0.6 | Glycerin | 1 |
| Additional polyol | Propylene | 0.6 | none | 0 |
| | glycol | | | |
| Chelating agent | DTPA | 0.2 | none | 0 |
| Organic acid | Dehydroacetic | 0.15 | Peroxyacetic | 0.2 |
| | acid | | acid | |
| pH adjuster | HCl or NaOH | q.s. for pH | HCl or | q.s. for pH |
| | | adjustment | NaOH | adjustment |
| Purified water | q.s. 100 | q.s. 100 | q.s. 100 | q.s. 100 |
| Expected pH | — | ~6-8 | — | ~6-8 |

Note:
⁽³⁾sodium alginate from FMC BioPolymer, G/M ratio of 30-35/65-70,
viscosity of 7-150 mPa · s.

| | Example | | | |
|---|---|---|---|---|
| Type of | 8 | | 9 | |
| Ingredient | Ingredient | (wt. %) | Ingredient | (wt. %) |
| Buffer | Citrate | 0.75 | Succinate | 1 |
| Alginate | Protanal LF | 0.3 | Protanal LF | 0.4 |
| | 120M⁽⁴⁾ | | 120M⁽⁴⁾ | |
| Polyol | Glycerin | 0.6 | Glycerin | 1 |
| Additional | Mannitol | 0.4 | Xylitol | 0.2 |
| polyol | | | | |
| Chelating agent | DTPA | 0.2 | NOTA | 0.3 |
| Organic acid | Dehydroacetic | 0.15 | Methylglutaric | 0.15 |
| | acid | | acid | |
| pH adjuster | HCl or NaOH | q.s. for pH | HCl or NaOH | q.s. for pH |
| | | adjustment | | adjustment |
| Purified water | q.s. 100 | q.s. 100 | q.s. 100 | q.s. 100 |
| Expected pH | — | ~6-8 | — | ~6-8 |

Note:
⁽⁴⁾sodium alginate from FMC BioPolymer, G/M ratio of 35-45/55-65,
viscosity of 7-150 mPa · s.

| | Example | | | |
|---|---|---|---|---|
| Type of | 10 | | 11 | |
| Ingredient | Ingredient | (wt. %) | Ingredient | (wt. %) |
| Buffer | Maleate | 1 | Phosphate | 1 |
| Alginate | Protanal LF | 0.3 | Protanal LF | 0.4 |
| | 120M⁽⁴⁾ | | 120M⁽⁴⁾ | |
| Polyol | Glycerin | 0.6 | Glycerin | 1 |
| Additional polyol | Sorbitol | 0.4 | Xylitol | 0.2 |
| Chelating agent | DO3A | 0.2 | NOTA | 0.3 |
| Organic acid | Propionic | 0.2 | Peroxyformic | 0.15 |
| | acid | | acid | |
| pH adjuster | HCl or | q.s. for pH | HCl or | q.s. for pH |
| | NaOH | adjustment | NaOH | adjustment |
| Purified water | q.s. 100 | q.s. 100 | q.s. 100 | q.s. 100 |
| Expected pH | — | ~6-8 | — | ~6-8 |

Note:
⁽⁴⁾see above.

| | Example | | | |
|---|---|---|---|---|
| Type of | 12 | | 13 | |
| Ingredient | Ingredient | (wt. %) | Ingredient | (wt. %) |
| Buffer | Maleate | 1 | Phosphate | 1 |
| Alginate | Protanal LF | 0.3 | Protanal LF | 0.4 |
| | 120M⁽⁴⁾ | | 120M⁽⁴⁾ | |
| Polyol | Glycerin | 0.6 | Glycerin | 1 |
| Additional polyol | Sorbitol | 0.4 | Xylitol | 0.2 |
| Chelating agent | none | 0 | none | 0 |
| Organic acid | sorbic acid | 0.2 | none | 0 |
| pH adjuster | HCl or NaOH | q.s. for pH | HCl or | q.s. for pH |
| | | adjustment | NaOH | adjustment |
| Purified water | q.s. 100 | q.s. 100 | q.s. 100 | q.s. 100 |
| Expected pH | — | ~6-8 | — | ~6-8 |

Note:
⁽⁴⁾see above.

In another aspect, the present invention provides a method for preparing an ophthalmic composition. The method comprises: (a) blending appropriate amounts of alginate and materials of a buffering system in a first sterilized vessel; (b) providing an amount of purified water equivalent to about 85-90 percent of the desired final weight of a batch in a second sterilized vessel; (c) heating the contents of the second vessel to about 45-50° C.; (d) stirring the contents of the second vessel for about 10-30 minutes, while maintaining the temperature; (e) adding appropriate amounts of polyol and other desired ingredients to the second vessel; (f) transferring the contents of the first vessel to the second vessel; (g) adding purified water to the second vessel in an amount sufficient to bring the batch to the desired total weight; (h) mixing the contents of the second vessel for about 0.5 to about 3 hours while maintaining the temperature; (i) cooling the contents of the second vessel to room temperature; (j) filtering the contents of the second vessel through a 0.2 μm filter to produce the ophthalmic composition. The composition is ready for packaging, storage, and use.

In one embodiment, a composition for reducing, ameliorating, treating, or preventing a condition of dry eye, the composition consists essentially of: (a) alginate in a concentration from about 0.01 to about 2 percent by weight of the total composition; (b) glycerin in a concentration from about 0.1 to about 1 percent by weight of the total composition; (c) propylene glycol in a concentration from about 0.1 to about 1 percent by weight of the total composition; (d) a buffering system or agent; (e) a chelating agent; and (f) water; wherein the composition has a pH from about 6.5 to about 7.5, and osmolality in a range from about 200 to about 240 mOsm/kg. In one embodiment, the chelating agent consists essentially of hydroxyalkylphosphonic acid, or DTPA, or EDTA, or a salt thereof. In another embodiment, the chelating agent is present in an amount from about 0.01 to about 0.2 percent by weight of the total composition. In still another embodiment, said buffering system or agent consists essentially of boric acid/borate buffer.

In another embodiment, a composition for reducing, ameliorating, treating, or preventing a condition of dry eye, the composition consists essentially of: (a) alginate in a concentration from about 0.1 to about 1 percent by weight of the total composition; (b) glycerin in a concentration from about 0.1 to about 1 percent by weight of the total composition; (c) propylene glycol in a concentration from about 0.1 to about 1 percent by weight of the total composition; (d) a buffering system or agent; and (e) water; wherein the composition has a pH from about 6.5 to about 7.5, and osmolality in a range from about 200 to about 240 mOsm/kg. In another embodiment, said buffering system or agent is boric acid/borate buffer.

In still another embodiment, a composition for reducing, ameliorating, treating, or preventing a condition of dry eye, the composition consists essentially of: (a) alginate in a concentration from about 0.1 to about 1 percent by weight of the total composition; (b) glycerin in a concentration from about 0.1 to about 1 percent by weight of the total composition; (c) propylene glycol in a concentration from about 0.1 to about 1 percent by weight of the total composition; (d) a buffering system or agent; (e) sorbic acid in a concentration from about 0.01 to about 1 percent by weight of the total composition; and (f) water; wherein the composition has a pH from about 6.5 to about 7.5, and osmolality in a range from about 200 to about 240 mOsm/kg. In another embodiment, said buffering system or agent is boric acid/borate buffer.

In still another embodiment, a composition for reducing, ameliorating, treating, or preventing a condition of dry eye, the composition consists essentially of: (a) alginate in a concentration from about 0.1 to about 1 percent by weight of the total composition; (b) glycerin in a concentration from about 0.1 to about 1 percent by weight of the total composition; (c) propylene glycol in a concentration from about 0.1 to about 1 percent by weight of the total composition; (d) a buffering system or agent; (e) sorbic acid in a concentration from about 0.01 to about 1 percent by weight of the total composition; (f) a chelating agent in a concentration from about 0.05 to about 0.2 percent by weight of the total composition; and (g) water; wherein the composition has a pH from about 6.5 to about 7.5, and osmolality in a range from about 200 to about 240 mOsm/kg. In another embodiment, said buffering system or agent is boric acid/borate buffer.

In yet another embodiment, a composition for reducing, ameliorating, treating, or preventing a condition of dry eye, the composition consists essentially of: (a) alginate in a concentration from about 0.1 to about 1 percent by weight of the total composition; (b) glycerin in a concentration from about 0.1 to about 1 percent by weight of the total composition; (c) propylene glycol in a concentration from about 0.1 to about 1 percent by weight of the total composition; (d) a buffering system or agent; (e) an organic acid in a concentration from about 0.01 to about 2 percent by weight of the total composition, said organic acid being selected from the group consisting of acetic acid, dehydroacetic acid, proprionic acid, butyric acid, isobutyric acid, valeric acid, hexanoic acid (caproic acid), heptanoic acid (enanthic acid), octanoic acid (caprylic acid), nonanoic acid (pelargonic acid), decanoic acid (capric acid), (+) camphoric acid, peroxyacetic acid, n-peroxybutyric acid, peroxyformic acid, peroxypropionic acid, malonic acid, dimethylmalonic acid, succinic acid, glutaric acid, β-methylglutaric acid, adipic acid, pimelic acid, suberic acid, azelaic acid, 1,1-cyclopentanediacetic acid, 1,2-trans-cyclopentanedicarboxylic acid, 1,3-trans-cyclopentanedicarboxylic acid, 1,3-trans-cyclohexanedicarboxylic acid, 1,4-cis-cyclohexanedicarboxylic acid, cyclohexanecarboxylic acid, benzoic acid, methoxybenzoic acid, p-n-propoxybenzoic acid, p-n-butoxybenzoic acid, and combinations thereof; and (f) water; wherein the composition has a pH from about 6.5 to about 7.5 and osmolality from about 200 to about 240 mOsm/kg. In another embodiment, said buffering system or agent is boric acid/borate buffer.

In a further embodiment, a composition for reducing, ameliorating, treating, or preventing a condition of dry eye, the composition consists essentially of: (a) alginate in a concentration from about 0.1 to about 1 percent by weight of the total composition; (b) glycerin in a concentration from about 0.1 to about 1 percent by weight of the total composition; (c) propylene glycol in a concentration from about 0.1 to about 1 percent by weight of the total composition; (d) a buffering system or agent; (e) an organic acid in a concentration from about 0.01 to about 2 percent by weight of the total composition, said organic acid being selected from the group consisting of acetic acid, dehydroacetic acid, proprionic acid, butyric acid, isobutyric acid, valeric acid, hexanoic acid (caproic acid), heptanoic acid (enanthic acid), octanoic acid (caprylic acid), nonanoic acid (pelargonic acid), decanoic acid (capric acid), (+) camphoric acid, peroxyacetic acid, n-peroxybutyric acid, peroxyformic acid, peroxypropionic acid, malonic acid, dimethylmalonic acid, succinic acid, glutaric acid, β-methylglutaric acid, adipic acid, pimelic acid, suberic acid, azelaic acid, 1,1-cyclopentanediacetic acid, 1,2-trans-cyclopentanedicarboxylic acid, 1,3-trans-cyclopentanedicarboxylic acid, 1,3-trans-cyclohexanedicarboxylic acid, 1,4-cis-cyclohexanedicarboxylic acid, cyclohexanecarboxylic acid, benzoic acid, methoxybenzoic acid, p-n-propoxybenzoic acid, p-n-butoxybenzoic acid, and combinations thereof; (f) a chelating agent consisting essentially of a hydroxyalkyl phosphonic acid in a concentration from about 0.005 to about 0.2 percent by weight of the total composition; and (g) water; wherein the composition has a pH from about 6.5 to about 7.5 and osmolality from about 200 to about 240 mOsm/kg. In another embodiment, said buffering system or agent is boric acid/borate buffer.

In another aspect, any one of the compositions of the present invention can be formed into a solution, an emulsion (such as an oil-in-water emulsion), a dispersion, a gelable composition, or a gel.

While specific embodiments of the present invention have been described in the foregoing, it will be appreciated by those skilled in the art that many equivalents, modifications, substitutions, and variations may be made thereto without departing from the spirit and scope of the invention as defined in the appended claims.

What is claimed is:

1. A composition comprising: (a) alginate; and (b) at least a polyol; wherein the composition has a pH in a range from about 5 to about 7.5 and osmolality in a range from about 200 to about 400 mOsm/kg.

2. The composition of claim 1, wherein the composition is essentially free of preservatives.

3. The composition of claim 2, further comprising a buffering system or agent.

4. The composition of claim 3, further comprising a chelating agent.

5. The composition of claim 4, wherein said alginate, polyol, and chelating agent are present a concentration from about 0.01 to about 2, from about 0.1 to about 1, and from about 0.005 to about 0.2 percent by weight of the total composition, respectively.

6. A composition for reducing, ameliorating, treating, or preventing a condition of dry eye, the composition consisting essentially of: (a) alginate in a concentration from about 0.01 to about 2 percent by weight of the total composition; (b) glycerin in a concentration from about 0.1 to about 1 percent by weight of the total composition; (c) propylene glycol in a concentration from about 0.1 to about 1 percent by weight of the total composition; (d) a buffering system or agent; (e) water; wherein the composition has a pH from about 6.5 to about 7.5 and osmolality in a range from about 200 to about 240 mOsm/kg.

7. A composition for reducing, ameliorating, treating, or preventing a condition of dry eye, the composition consisting essentially of: (a) alginate in a concentration from about 0.01 to about 2 percent by weight of the total composition; (b) glycerin in a concentration from about 0.1 to about 1 percent by weight of the total composition; (c) propylene glycol in a concentration from about 0.1 to about 1 percent by weight of the total composition; (d) a buffering system or agent; (e) a chelating agent consisting essentially of hydroxyalkylphosphonic acid, or DTPA, or EDTA, or a salt thereof; and (f) water; wherein the composition has a pH from about 6.5 to about 7.5 and osmolality in a range from about 200 to about 240 mOsm/kg.

8. The composition of claim 7, wherein said buffering system or agent consists essentially of boric acid and sodium borate buffer.

9. A composition for reducing, ameliorating, treating, or preventing a condition of dry eye, the composition consisting essentially of: (a) alginate in a concentration from about 0.1 to about 1 percent by weight of the total composition; (b) glycerin in a concentration from about 0.1 to about 1 percent by weight of the total composition; (c) propylene glycol in a concentration from about 0.1 to about 1 percent by weight of the total composition; (d) a boric acid and sodium borate buffering system; (e) sorbic acid in a concentration from about 0.01 to about 1 percent by weight of the total composition; and (f) water; wherein the composition has a pH from about 6.5 to about 7.5, and osmolality in a range from about 200 to about 240 mOsm/kg.

10. A composition for reducing, ameliorating, treating, or preventing a condition of dry eye, the composition consisting essentially of: (a) alginate in a concentration from about 0.1 to about 1 percent by weight of the total composition; (b) glycerin in a concentration from about 0.1 to about 1 percent by weight of the total composition; (c) propylene glycol in a concentration from about 0.1 to about 1 percent by weight of the total composition; (d) a buffering system or agent; (e) sorbic acid in a concentration from about 0.01 to about 1 percent by weight of the total composition; (f) a chelating agent in a concentration from about 0.05 to about 0.2 percent by weight of the total composition; and (g) water; wherein the composition has a pH from about 6.5 to about 7.5, and osmolality in a range from about 200 to about 240 mOsm/kg.

11. The composition of claim 10, wherein said buffering system or agent consists essentially of a boric acid and borate buffer.

12. A composition for reducing, ameliorating, treating, or preventing a condition of dry eye, the composition consisting essentially of: (a) alginate in a concentration from about 0.1 to about 1 percent by weight of the total composition; (b) glycerin in a concentration from about 0.1 to about 1 percent by weight of the total composition; (c) propylene glycol in a concentration from about 0.1 to about 1 percent by weight of the total composition; (d) a buffering system or agent; (e) an organic acid in a concentration from about 0.01 to about 2 percent by weight of the total composition, said organic acid being selected from the group consisting of acetic acid, dehydroacetic acid, proprionic acid, butyric acid, isobutyric acid, valeric acid, hexanoic acid (caproic acid), heptanoic acid (enanthic acid), octanoic acid (caprylic acid), nonanoic acid (pelargonic acid), decanoic acid (capric acid), (+) camphoric acid, peroxyacetic acid, n-peroxybutyric acid, peroxyformic acid, peroxypropionic acid, malonic acid, dimethylmalonic acid, succinic acid, glutaric acid, β-methylglutaric acid, adipic acid, pimelic acid, suberic acid, azelaic acid, 1,1-cyclopentanediacetic acid, 1,2-trans-cyclopentanedicarboxylic acid, 1,3-trans-cyclopentanedicarboxylic acid, 1,3-trans-cyclohexanedicarboxylic acid, 1,4-cis-cyclohexanedicarboxylic acid, cyclohexanecarboxylic acid, benzoic acid, methoxybenzoic acid, p-n-propoxybenzoic acid, p-n-butoxybenzoic acid, and combinations thereof; and (f) water; wherein the composition has a pH from about 6.5 to about 7.5 and osmolality from about 200 to about 240 mOsm/kg.

13. A composition for reducing, ameliorating, treating, or preventing a condition of dry eye, the composition consisting essentially of: (a) alginate in a concentration from about 0.1 to about 1 percent by weight of the total composition; (b) glycerin in a concentration from about 0.1 to about 1 percent by weight of the total composition; (c) propylene glycol in a concentration from about 0.1 to about 1 percent by weight of the total composition; (d) a buffering system or agent; (e) an organic acid in a concentration from about 0.01 to about 2 percent by weight of the total composition, said organic acid being selected from the group consisting of acetic acid, dehydroacetic acid, proprionic acid, butyric acid, isobutyric acid, valeric acid, hexanoic acid (caproic acid), heptanoic acid (enanthic acid), octanoic acid (caprylic acid), nonanoic acid (pelargonic acid), decanoic acid (capric acid), (+) camphoric acid, peroxyacetic acid, n-peroxybutyric acid, peroxyformic acid, peroxypropionic acid, malonic acid, dimethylmalonic acid, succinic acid, glutaric acid, β-methylglutaric acid, adipic acid, pimelic acid, suberic acid, azelaic acid, 1,1-cyclopentanediacetic acid, 1,2-trans-cyclopentanedicarboxylic acid, 1,3-trans-cyclopentanedicarboxylic acid, 1,3-trans-cyclohexanedicarboxylic acid, 1,4-cis-cyclohexanedicarboxylic acid, cyclohexanecarboxylic acid, benzoic acid, methoxybenzoic acid, p-n-propoxybenzoic acid, p-n-butoxybenzoic acid, and combinations thereof; (f) a chelating agent consisting essentially of a hydroxyalkyl phosphonic acid in a concentration from about 0.005 to about 0.2 percent by weight of the total composition; and (g) water; wherein the composition has a pH from about 6.5 to about 7.5 and osmolality from about 200 to about 240 mOsm/kg. In another embodiment, said buffering system or agent is boric acid/borate buffer.

14. A method for reducing, ameliorating, treating, or preventing a condition of dry eye, the method comprising administering to an eye affected by said condition a composition that comprises: (a) alginate; and (b) at least a polyol; wherein the composition has a pH in a range from about 5 to about 7.5.

15. The method of claim 14, wherein the composition is essentially free of preservatives, and further comprises a buffering system or agent and a chelating agent, wherein said alginate, polyol, and chelating agent are present a concentration from about 0.01 to about 2, from about 0.1 to about 1, and from about 0.005 to about 0.2 percent by weight of the total composition, respectively.

16. A method for reducing, ameliorating, treating, or preventing a condition of dry eye, the method comprising administering to an eye affected by said condition a composition that consists essentially of: (a) alginate in a concentration from about 0.1 to about 1 percent by weight of the total composition; (b) glycerin in a concentration from about 0.1 to about 1 percent by weight of the total composition; (c) propylene glycol in a concentration from about 0.1 to about 1 percent by weight of the total composition; (d) a boric acid and sodium borate buffering system; (e) sorbic acid in a concentration from about 0.01 to about 1 percent by weight of the total composition; and (f) water; wherein the composition has a pH from about 6.5 to about 7.5, and osmolality in a range from about 200 to about 240 mOsm/kg.

17. A method for reducing, ameliorating, treating, or preventing a condition of dry eye, the method comprising administering to an eye affected by said condition a composition that consists essentially of: (a) alginate in a concentration from about 0.1 to about 1 percent by weight of the total composition; (b) glycerin in a concentration from about 0.1 to about 1 percent by weight of the total composition; (c) propylene glycol in a concentration from about 0.1 to about 1 percent by weight of the total composition; (d) a buffering system or agent; (e) sorbic acid in a concentration from about 0.01 to about 1 percent by weight of the total composition; (f) a chelating agent in a concentration from about 0.05 to about 0.2 percent by weight of the total composition; and (g) water; wherein the composition has a pH from about 6.5 to about 7.5, and osmolality in a range from about 200 to about 240 mOsm/kg.

18. The method of claim 17, wherein said buffering system or agent consists essentially of a boric acid and borate buffer.

19. A method for reducing, ameliorating, treating, or preventing a condition of dry eye, the method comprising administering to an eye affected by said condition a composition that consists essentially of: (a) alginate in a concentration from about 0.1 to about 1 percent by weight of the total composition; (b) glycerin in a concentration from about 0.1 to about 1 percent by weight of the total composition; (c) propylene glycol in a concentration from about 0.1 to about 1 percent by weight of the total composition; (d) a buffering system or agent; (e) an organic acid in a concentration from about 0.01 to about 2 percent by weight of the total composition, said organic acid being selected from the group consisting of acetic acid, dehydroacetic acid, proprionic acid, butyric acid, isobutyric acid, valeric acid, hexanoic acid (caproic acid), heptanoic acid (enanthic acid), octanoic acid (caprylic acid), nonanoic acid (pelargonic acid), decanoic acid (capric acid), (+) camphoric acid, peroxyacetic acid, n-peroxybutyric acid, peroxyformic acid, peroxypropionic acid, malonic acid, dimethylmalonic acid, succinic acid, glutaric acid, β-methylglutaric acid, adipic acid, pimelic acid, suberic acid, azelaic acid, 1,1-cyclopentanediacetic acid, 1,2-trans-cyclopentanedicarboxylic acid, 1,3-trans-cyclopentanedicarboxylic acid, 1,3-trans-cyclohexanedicarboxylic acid, 1,4-cis-cyclohexanedicarboxylic acid, cyclohexanecarboxylic acid, benzoic acid, methoxybenzoic acid, p-n-propoxybenzoic acid, p-n-butoxybenzoic acid, and combinations thereof; and (f) water; wherein the composition has a pH from about 6.5 to about 7.5 and osmolality from about 200 to about 240 mOsm/kg.

20. A method for reducing, ameliorating, treating, or preventing a condition of dry eye, the method comprising administering to an eye affected by said condition a composition that consists essentially of: (a) alginate in a concentration from about 0.1 to about 1 percent by weight of the total composition; (b) glycerin in a concentration from about 0.1 to about 1 percent by weight of the total composition; (c) propylene glycol in a concentration from about 0.1 to about 1 percent by weight of the total composition; (d) a buffering system or agent; (e) an organic acid in a concentration from about 0.01 to about 2 percent by weight of the total composition, said organic acid being selected from the group consisting of acetic acid, dehydroacetic acid, proprionic acid, butyric acid, isobutyric acid, valeric acid, hexanoic acid (caproic acid), heptanoic acid (enanthic acid), octanoic acid (caprylic acid), nonanoic acid (pelargonic acid), decanoic acid (capric acid), (+) camphoric acid, peroxyacetic acid, n-peroxybutyric acid, peroxyformic acid, peroxypropionic acid, malonic acid, dimethylmalonic acid, succinic acid, glutaric acid, β-methylglutaric acid, adipic acid, pimelic acid, suberic acid, azelaic acid, 1,1-cyclopentanediacetic acid, 1,2-trans-cyclopentanedicarboxylic acid, 1,3-trans-cyclopentanedicarboxylic acid, 1,3-trans-cyclohexanedicarboxylic acid, 1,4-cis-cyclohexanedicarboxylic acid, cyclohexanecarboxylic acid, benzoic acid, methoxybenzoic acid, p-n-propoxybenzoic acid, p-n-butoxybenzoic acid, and combinations thereof; (f) a chelating agent consisting essentially of a hydroxyalkyl phosphonic acid in a concentration from about 0.005 to about 0.2 percent by weight of the total composition; and (g) water; wherein the composition has a pH from about 6.5 to about 7.5 and osmolality from about 200 to about 240 mOsm/kg. In another embodiment, said buffering system or agent is boric acid/borate buffer.
